SEQUENCE LISTING (4 sequences)

SEQ ID NO: 1 - protein, 123 aa, Unknown
  synthetic
AAALDQAKQAALKEFDRYGVSNYYKNLINKAKTVEGIMELQAQVVESAKKARISEATDGLSDFLKSQTSAEDTLKSIKLSEAKEMAIRELDAQGVSDFYKNKINNAKTVEGVVALKDLILNSL

SEQ ID NO: 2 - DNA, 369 bp, Unknown
  synthetic
gctgctgctttagatcaagctaagcaagctgctctaaaagaattcgatcgttatggtgtgagcaactactataaaaatcttattaataaagctaaaactgttgaaggaattatggagcttcaggcacaagttgttgagtcagccaaaaaagcacgtatttcagaggcaacagatggtttatctgatttcttgaagtctcaaacttcagcagaagacacccttaaatcaattaaactttcagaagctaaggagatggctattcgtgagttagatgctcagggtgttagtgacttttataagaacaaaattaacaatgctaagactgtagaaggagttgttgcgcttaaagaccttattcttaactcctta

SEQ ID NO: 3 - protein, 440 aa, Unknown
  synthetic
LGIDPFTALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTESRALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEKGELKLCITGDALVALPEGES

SEQ ID NO: 4 - DNA, 1320 bp, Unknown
  synthetic
ctgggaattgatcccttcaccgcattacctaagactgacacttacaaattaatccttaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaaccagaagtgatcgatgcgtctgaattaacaccagccgtgacaacttacaaacttgttattaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaaccagaagtgatcgatgcgtctgaattaacaccagccgtgacaacttacaaacttgttattaatggtaaaacattgaaaggcgaaacaactactaaagcagtagacgcagaaactgcagaaaaagccttcaaacaatacgctaacgacaacggtgttgatggtgtttggacttatgatgatgcgactaagacctttacggtaactgaaatggttacagagtctagagcattacctaagactgacacttacaaattaatccttaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaaccagaagtgatcgatgcgtctgaattaacaccagccgtgacaacttacaaacttgttattaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaaccagaagtgatcgatgcgtctgaattaacaccagccgtgacaacttacaaacttgttattaatggtaaaacattgaaaggcgaaacaactactaaagcagtagacgcagaaactgcagaaaaagccttcaaacaatacgctaacgacaacggtgttgatggtgtttggacttatgatgatgcgactaagacctttacggtaactgaaatggttacagagaagggcgagctcaagctttgcatcacgggagatgcactagttgccctacccgagggcgagtcg

(19) United States
(12) Patent Application Publication
(10) Pub. No.: US 2013/0066046 A1
(43) Pub. Date: Mar. 14, 2013

(54) General Method for Generating Ultra-High Affinity Binding Proteins

(71) Applicants: Roy H. Hammerstedt, Boalsburg, PA (US); Michael D. Boyle, Nelsonville, OH (US)

(72) Inventors: Roy H. Hammerstedt, Boalsburg, PA (US); Michael D. Boyle, Nelsonville, OH (US)

(21) Appl. No.: 13/513,373

(22) PCT Filed: Dec. 2, 2010
(86) PCT No.: PCT/US2010/003076

§ 371 Date: Jun. 13, 2012

Related U.S. Application Data
(60) Provisional application No. 61/283,417, filed on Dec. 3, 2009.

(51) Int. Cl. C12P 21/02; C07K 14/00

(52) U.S. Cl. 530/300; 435/71.2; 530/350

(57) ABSTRACT

A method for combining two or more maximum moieties to yield functional cassettes with ultra-high binding affinities, in which at least two binding sequences are incorporated into the same linear polypeptide such that each is separated by between five and forty amino acids, to create a polypeptide having geometrically increased binding affinity compared to the arithmetically summed binding affinities of the individual maximum moieties.

### STATEMENT OF FEDERALLY FUNDED RESEARCH

The invention described herein was funded in part by a grant from the National Cancer Institute, The National Institutes of Health, Grant No. R43 CA124275-01. The United States Government has certain rights to the invention.

### BACKGROUND OF THE INVENTION

1. Field of the Invention

The invention is directed to reproducible molecules having ultra high binding affinities, and methods of making them. Binding proteins found within and on cells have been recognized for decades, and many have been developed to commercial utility. The development of methods to combine the selection of unique reproducible amino acid sequences that occur naturally and subsequently to deploy the affinity of the those amino acid sequences for enhanced utility provided by a single polypeptide chain is the focus of this patent application.

2. Description of Related Art

In the 50 years since the identification of the existence of a surface antigen on S aureus that reacted with all 500 human serum samples tested, independent of a subject's exposure to the organism, many different “non immune” reactions between bacteria and plasma have been described. These and other empirical observations extended the concept of “surface binding proteins for serum components” to many gram positive and gram negative bacteria beyond the originally defined S aureus.

As one example, a host of distinct binding properties have been identified (FIG. 1) on various isolates of S pyrogenes. The majority of these genes have been cloned and expressed as functional molecules in E coli, setting the stage for manipulation by molecular biological techniques to yield recombinant forms or fragments of the native protein which have shown utility for a number of defined purposes.

Ultra-high binding affinities (Kd values on the order of 10E-14M) are useful attributes, but are rare. A prototype natural material is the biotin-(strept)avidin system where the four individual peptide chains associate, without covalent attachment, to provide the necessary conformation for ultra-high binding.

A scheme for identifying the existence of desirable activities is presented (FIGS. 2 and 3) which has been used successfully in prior art to detect functional binding proteins (for examples see Bacterial immunoglobulin-binding proteins volume I and II Boyle (editor) Academic Press, 1990).

An approach illustrating prior art used in an attempt to get to ultra-high affinities is reflected in FIG. 3. The experimental path begins with flow through FIG. 1 and then FIG. 2, leading to the identification of the amino acid sequence containing the total binding activity for the target ligand within that single polypeptide chain. Note that this isolated polypeptide chain can contain several amino acid sequences, not necessarily identical, that share the common property of binding to one or more target ligand(s). This is defined as the maximum moiety. In those cases where several sequences in the same chain had binding activity, or amino acids without binding activity are identified, in the reductive approach they excised components from the maximum moiety until they had a simpler sequence with a minimal amino acid sequence with the desired binding features. This is termed the minimum moiety. Then a variety of methods are already known to manipulate the composition of key amino acids in a critical region(s) of the minimum moiety to increase binding. Jonsson et al. 2008 (Jonsson A, Dogan J, Herne N, Abrahmsen L and Nygren P-A, “Engineering of a femtomolar affinity binding protein to human serum albumin Protein Engineering,” Design & Selection, 21:515-527, 2008) provide an excellent example of combining targeted combinatorial protein engineering strategy to identify variants showing improved properties. They use a multi-staged, combinatorial-chemistry, phage-display approach to identify a final molecule, from millions of potential candidates, with a binding affinity in the ultra-high category, testing and optimizing fifteen individual amino acids to attain the final product. This example, plus those examples involved in standard methods encompassed under the names of affinity maturation, molecular evolution, and combinatorial chemistry, represent a randomized approach. In general, one can expect 10-100× increase in binding affinity at each stage of screening, but the output illustrates that with enough separate stages very impressive improvements can be achieved.

Alternative approaches in the prior art to create ultra-high binding affinities have been described. Borman 2002 (Borman S., “Multivalency: Strength in Numbers,” Chemical and Engineering News, Oct. 9, 2002, pp. 48-53) summarized the concept of multi-valency, where there is the simultaneous attachment of two or more binding sites on one biological molecule to multiple receptors sites on another. Exploitation of multiple, pre-planned, weak binding activities can yield strong attachments through features of both receptor(s) and ligand(s).

In nature, the binding activities can be mapped to a fragment of the entire protein. Distinct regions of the protein may contain one or more closely repeated amino acid sequences that can participate in ligand binding, e.g. staphylococcal protein A (Langone, J J., “Protein A of Staphylococcus aureus and related immunoglobulin receptors produced by streptococci and pneumonococci,” Adv Immunol., 32:157-252 (1982)) protein L (Housden N G, Harrison S, Roberts S E, Beckingham J A, Graille M, Stura E, Gore M G, “Immunoglobulin-binding domains: Protein L Peptostreptococcus magnus,” Biochem Soc Trans., 31:716-718 (2003)) or streptococcal protein G (Fahnestock S R, Alkexander P, Filpula D, Nagle J, “Structure and evolution of the streptococcal genes encoding protein G,” in Bacterial Immunoglobin-binding proteins, Vol 1 (Boyle MDP editor) Academic Press, New York, pp 49-70 (1990)). The number of naturally occurring repeat regions has been shown to increase the binding affinity in a proportional manner with an approximate 10× increase in affinity for each additional domain (Fahnestock et al. 1990) or have minimal changes in affinity, as when a protein A/G chimera is generated (Eliasson M, Olsson A, Palmcrantz E, Wiberg K, Inganäs M, Guss B, Lindberg M, and Uhlén M., “Chimeric IgG-binding receptors engineered from staphylococcal protein A and streptococcal protein G,” J. Biol. Chem., 263:4323-4327 (1988)).

The analogy of lock and key is a time-tested approach for description of binding in biological systems, and is used herein for the example of interest.

The goal of any binding affinity assay to precisely measure the “goodness-of-fit” between the components of “lock” (e.g., binding protein) and the “key” (the target; e.g., albumin) and is complicated by the nature of the assays used to make the measurements. In the mechanical world, the lock and key can be made to high tolerance with materials-of-choice, which allows a precise description of their interaction. In the biological world, the nature of biopolymers precludes any such precise description because, while biochemical activity might be attributed to a few key amino acids, the broader surrounding environment (i.e. auxiliary residues in spatial proximity) often plays an equally import role. The following series of analogies describes the limits to any assay, all of which are related to the need to modify some aspect of the lock, the key, or both to allow measurement of the concentration of the components of interest as they interact.

First, examples of the key are presented: the key is often converted to a detectably labeled derivative. The key could be chemically synthesized (and thus of completely known structure) or it could isolated from biological systems (with unknown numbers of subspecies; e.g., glycosylated albumin that might or might not bind). To simplify measurement, common strategies could involve covalent attachment of a “hook” (e.g., biotin) to allow secondary attachment of additional molecules to facilitate measurement (e.g., streptavidin-peroxidase), or perhaps a “light” (fluorescein) to facilitate finding (e.g., spectroscopic detection) the key. While useful, those skilled in the art understand that the physical approach of the key to the lock can be greatly altered by the number and nature of both the presence of subspecies and any secondary appendages, and that this will alter—in unpredictable ways—the numerical values extracted with the assay.

Next, examples of the lock are presented. The tumbler of the lock (the few amino acids describing the points of contact) is itself not rigid, and the fact that neither the door, the wall nor the building is rigid further complicates the situation. The net effect is that the tumbler performs in context of the entirety of the network of proteins and other macromolecules of the system. In context of this system, neither the functional cassette nor additional protein components added to facilitate commercialization are rigid.

Further, a critical aspect of the assay is to effect separation free from bound target molecules, which often involves attachment of either the lock or the key to a solid surface (e.g., a bead or planar surface) which further affects approach of binding partners.

Finally, to facilitate the assay and to optimize opportunity to display features of interest, a complex system with multiple locks often is set up so that the key is distributed between each of the unique locks.

The sum of these factors leads to the well accepted fact that absolute numerical value (e.g., the dissociation constant) of interest estimated in one assay is not expected to be identical to one in an alternative assay. The numerical value can vary several-hundred-fold across the various assays available. Those skilled-in-the-art often assume that an assay free of complicating factors (i.e., minimally modified lock and/or key) that utilizes direct measurements is most likely to yield a best estimate of the “true Kd.”

An example of such variation is provided (FIG. 6 of Boyle MDP, editor, Bacterial Immunoglobin-binding Proteins Vol 1 & 2, Academic Press, New York (1990)) to illustrate the complicated nature of competitive assays. They tested forms of tracer known to differ in binding affinity, and demonstrated assay conditions such that there was virtually no difference in inhibition curves for one assay yet major differences were observed in another assay. This was interpreted by them to indicate that it is possible to take molecules with known differences in affinity and have them yield superimposable inhibition curves.

These factors become important when decisions are made on the final format to be used for the commercial product. The final commercial form of a binding protein, chosen to satisfy additional factors such as cost, ease of use, etc., presents the functional cassette in its total context, will define the protein environment and thus final binding affinity.

Binding constants (indicated with capital K) are equilibrium constants and relate to this relationship; A+B→AB, where A and B are two species that can interact in a non-covalent manner to create a complex. We will consider the case where A is the dominant [higher concentration] species relative to B, and A is to be fixed in concentration over the time of interest. The binding constant relating the concentrations of the reactants (in molar units, M) can be expressed in either of two forms: (a) the association constant [Kₐ=[AB]/[A] [B]; units of M⁻¹]; or (b) the dissociation constant [Kd=[A] [B]/[AB]; units of M]. An alternative formulation to describe the binding constant is through use of the ratio of the velocities of the forward [velocity=kon [A][B]] and reverse [velocity=koff [AB]] reactions.

The numerical value of Kd dictates the amount of B that can bind to A at equilibrium. The velocities that allow achievement of that equilibrium is influenced by the kon and koff rates.

Since the concentration of A (e.g. albumin) is considered as large and fixed, the forward velocity is dictated by: (a) the concentration of B (the ligand, which might (does) vary over time); and (b) the kon (which reflects the diffusion rate which leads to a collision of A and B) and has the units of (M)⁻¹(sec)⁻¹. The speed at which B binds to A is greatly influenced by the concentration of B at that time. The velocity of the reverse reaction, in which the AB complex dissociates, is the product of AB and koff, which has units of (sec)⁻¹. At equilibrium, the pool of AB is constantly replenished from the A and B via the forward reaction, but in the case where B concentration approaches zero, the breakup of AB is first order and is often described by the time to lose half the bound materials [half-life]=ln 2/koff=0.69/koff.

Thus, if the AB complex is put in a situation where B is allowed to decrease (as when surrounding fluids are depleted of B), B dissociates from the complex. Absent B, reformation of AB is greatly reduced. If you want to trap a complex AB to reflect the equilibrium state, the koff becomes a dominant feature for consideration.

These concepts lead to the need to keep track of two different aspects of any equilibrium reaction; the Kd to indicate the position of equilibrium, and the koff or half-life to dictate the speed with which you must act to get an accurate estimate. Note that two systems might have the same Kd (e.g., 1×10 E-6 M) but can accomplish that equilibrium position by widely different forward and reverse velocities. The end Kd can be achieved with the ratio of forward and reverse velocities of either: (a) 10 E-7/10 E-1, with a t_{1/2} of 6.9 seconds; or (b) 10 E-9/10 E-3, with a t_{1/2}, of 690 seconds. This clearly indicates that what you find when you look for a AB complex is dictated by how fast you can accomplish the separation of that complex. Thus, in the absence of a direct measure of the various velocities, measurement of a Kd by an indirect method does not suitably predict the critical aspects of the equilibrium.

While applying these basic principles to a model system for human serum albumin we made an unexpected finding that the enhancement of affinity can be achieved by an additive approach, where increasing the number of maximum moieties as isolated per FIG. 3—in a single polypeptide chain—resulted in an unexpected and unpredicted enhancement of affinity to a level approaching that of biotin and avidin.

### SUMMARY OF THE INVENTION

The present invention is a method for combining two or more maximum moieties to yield functional cassettes with ultra-high binding affinities. A maximum moiety is a description of the biological activity related to the minimal sequence that accounts for all the binding activity of the intact protein for the target ligand as isolated from the source organism. The maximum moiety may contain individual amino acid sequences that differ in binding activity and are separated by spacers. A functional cassette is a plurality (two or more) of maximum moieties, which may be separated by spacers, and which may have termini that provide additional valuable features, including but not limited to aspects such as optimization of manufacture by recombinant technologies, increased solubility of final protein, enhanced and oriented binding to solid surfaces, or simplification of the processes needed in the formation of detectable labeled materials with ultra-high binding affinities. Therefore, the invention is a method of making functional cassettes, comprising: (a) identifying a maximum moiety of interest; (b) replicating a peptide or protein encoding said maximum binding moiety at least two times, to create at least two largely similar or identical binding sequences; and (c) coupling said at least two binding sequences (preferably three and more preferably 4 or more) into the same polypeptide such that each is separated by between five and forty amino acids, wherein the polypeptide has a geometrically increased binding affinity compared to the arithmetically summed binding affinities of the individual maximum moieties and further wherein the geometrically increased binding affinity yields a binding constant [Kd] of less than 1E-11 M with a half life of at least 36,000 seconds when the binding constant is determined by surface plasmon resonance assessment in real time. The invention is also a molecule generally made from the method, namely, a molecule containing two maximum moieties of interest embodied in two respective largely similar or identical binding sequences, with the binding sequences' being coupled into the same polypeptide separated by between five and forty amino acids, such that the molecule has a binding constant [Kd] of less than 1E-11M with a half life of at least 36,000 seconds when the binding constant is determined by surface plasmon resonance assessment in real time.

### BRIEF DESCRIPTION OF THE DRAWINGS

FIG. 1: Schematic representation of surface proteins of S pyogenes.

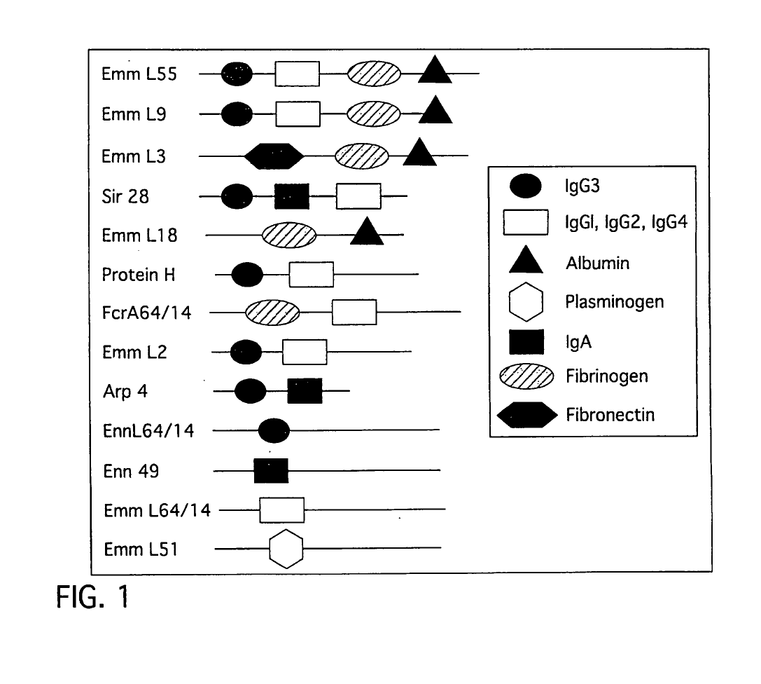

FIG. 2: Schematic of approach to detection of maximum moieties of binding activities; bacteria are used as one example of many different organisms.

FIG. 3: Expression and characterization of maximum moieties leading to identification and isolation of a minimum moiety.

FIG. 4: Schematic of one possible means of maximum moiety manipulation to yield a functional cassette as claimed herein.

FIG. 5: Representative BIACORE data set for binding construct “1” (with a single maximum moiety) to immobilized human serum albumin; after 1800 seconds of equilibration, sample was injected at indicated concentration over 300 seconds, followed by 2400 seconds of dissociation in buffer alone; data were evaluated using BIACORE software BIAevaluation VS 4.1.

FIG. 6: Competitive binding data for recombinant proteins with different amino acid sequences for binding of human serum albumin; Construct “1” represents competition between soluble and immobilized forms of a single maximum moiety.

FIG. 7: Data from example of extraction of human serum albumin with construct “2.”

FIG. 8: Data example of extraction of IgG from human serum.

### DETAILED DESCRIPTION OF THE INVENTION

As summarized above, the present invention is a method for combining two or more maximum moieties to yield functional cassettes with ultra-high binding affinities. A maximum moiety is a description of the biological activity related to the minimal sequence that accounts for all the binding activity of the intact protein for the target ligand as isolated from the source organism. The maximum moiety may contain individual amino acid sequences that differ in binding activity and are separated by spacers. A functional cassette is a plurality (two or more) of maximum moieties, which may be separated by spacers, and which may have termini that provide additional valuable features, including but not limited to aspects such as optimization of manufacture by recombinant technologies, increased solubility of final protein, enhanced and oriented binding to solid surfaces, or simplification of the processes needed in the formation of detectable labeled materials with ultra-high binding affinities. Therefore, the invention is a method of making functional cassettes, comprising: (a) identifying a maximum moiety of interest; (b) replicating a peptide or protein encoding said maximum binding moiety at least two times, to create at least two largely similar or identical binding sequences; and (c) coupling said at least two binding sequences (preferably three and more preferably 4 or more) into the same polypeptide such that each is separated by between five and forty amino acids, wherein the polypeptide has a geometrically increased binding affinity compared to the arithmetically summed binding affinities of the individual maximum moieties and further wherein the geometrically increased binding affinity yields a binding constant [Kd] of less than 1E-11 M with a half life of at least 36,000 seconds when the binding constant is determined by surface plasmon resonance assessment in real time. The invention is also a molecule generally made from the method, namely, a molecule containing two maximum moieties of interest embodied in two respective binding sequences, with the binding sequences' being coupled into the same polypeptide separated by between five and forty amino acids, such that the molecule has a binding constant [Kd] of less than 1E-11M with a half life of at least 36,000 seconds when the binding constant is determined by surface plasmon resonance assessment in real time.

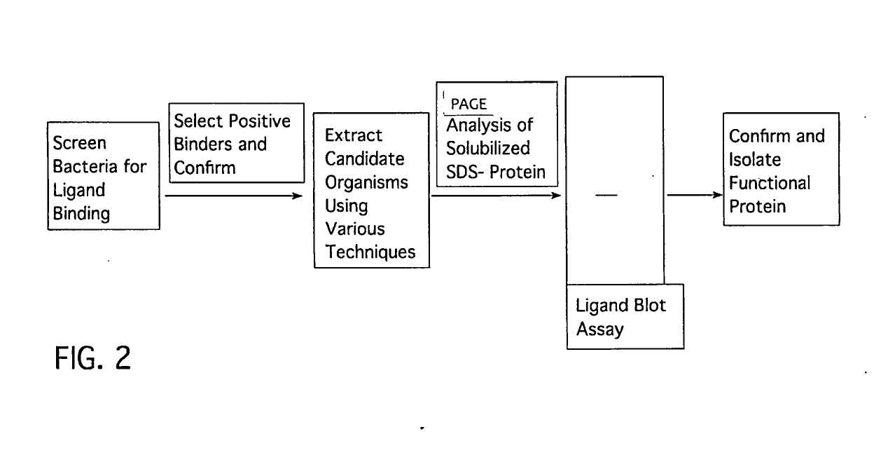

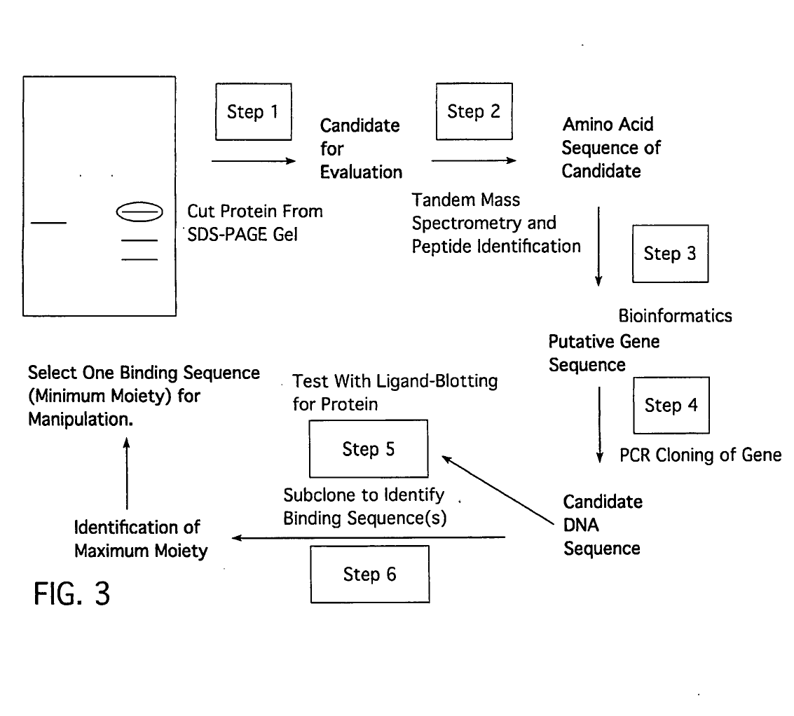

In a first example of the proposed invention a maximum moiety for human serum albumin is described, followed by illustrations of its binding features in several different assays. In a series of initial screening a number of group G bovine streptococcal isolates that bound human serum albumin were identified. The isolates BG1 and BG8 were used to evaluate the approach of FIG. 2. Both of these isolates were found reproducibly to bind high quantities of human serum albumin in direct binding and colony binding assays. Extracts of BG1 and BG8 were found to contain human serum albumin binding activity using a ligand blotting assay and both isolates demonstrated a similar pattern of active bands. This provided the starting point to initiate the series of studies outlined in FIG. 3.

A protein band in the hot acid extract of BG1 or BG8, corresponding on a parallel ligand-blotting gel that bound human serum albumin, was cut from an SDS-polyacrylamide gel and subjected to sequence analysis using tandem mass spectrometry. The results obtained from each isolate were similar; consequently, for all of the subsequent analysis results for only isolate BG8 are presented. Tandem mass spectral analysis of the albumin-binding band from BG8 identified three major peptides. Using Mascot algorithms, three potential matches with Mowse scores >100 were obtained. The highest score [FIG. 3, step 3] with a value of 203 and based on three peptide matches [ELDAQGVSDFYK, ISEATDGLSDFLK and TVEGIMELQAQVVE] was to an albumin-binding protein precursor from S canis. PCR primers based on the published partial gene sequence for the S. canis albumin-binding protein led to the amplification of a 1 kB gene sequence (FIG. 3, step 4). When incorporated into a suitable expression vector (pBAD) a recombined protein that bound albumin was expressed (FIG. 3, step 5). The cloned gene encoding an albumin binding protein derived following the outline of experiments was sequenced and used as the starting point for the studies (FIG. 3, step 5 and 6). The candidate gene was further subcloned (FIG. 3, step 6).

The DNA and protein structures of the maximum moiety for human serum albumin are provided. The amino acid sequence, encompassing two sequences differing in apparent affinity for human serum albumin (as assessed in Western blot studies), is A A A L D Q A K Q A A L K E F D R Y G V S N Y Y K N L I N K A K T V E G I M E L Q A Q V V E S A K K A R I S E A T D G L S D F L K S Q T S A E D T L K S I K L S E A K E M A I R E L D A Q G V S D F Y K N K I N N A K T V E G V V A L K D L I L N S L (SEQ ID NO. 1). The amino acids presented in bold print are sequences binding albumin (note different amino acid sequences), while those in regular case capitals are spacer regions. The DNA sequence covering the same regions is get get get tta gat caa get aag caa get get cta aaa gaa ttc gat cgt tat at ggt gtg agc aac tac tat at aaa aat ctt att aat aaa get aaa act gtt gaa gga att atg gag ctt cag gca caa gtt gtt gag tca gcc aaa aaa gca cgt att tca gag gca aca gat ggt tta tct gat ttc ttg aag tct caa act tca gca gaa gac acc ctt aaa tca att aaa ett tca gaa get aag gag atg get att cgt gag tta gat get cag ggt gtt agt gac ttt tat at aag aac aaa att aac aat get aag act gta gaa gga gtt gtt gcg ctt aaa gac ctt att ctt aac tcc tta (SEQ ID NO. 2).

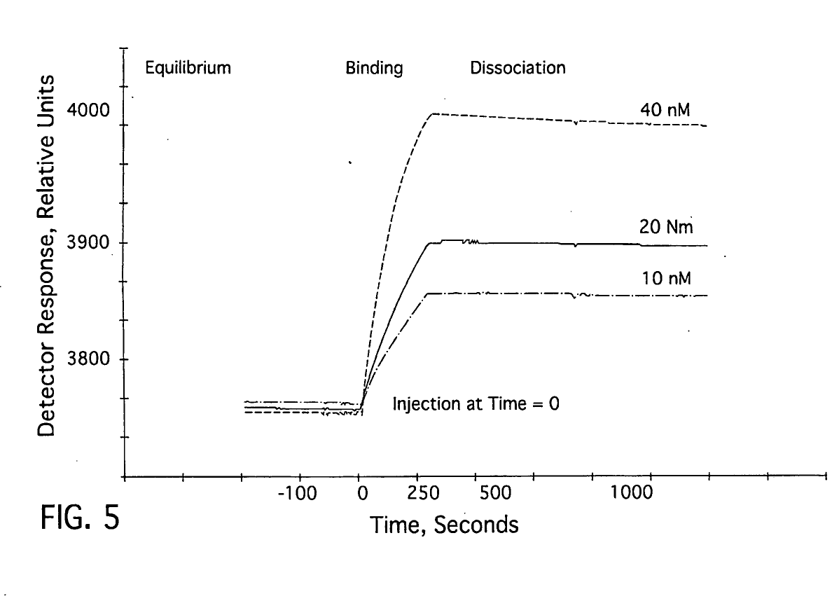

A further illustration 1.1—using the protocol of Gulich S, Linhult M, Nygren P-A, Uhlen M and Hober S J, “Stability towards alkaline conditions can be engineered into a protein ligand,” J. Biotechnol 80: 169-178 (2000) as described to assess binding affinities via Biacore technologies—provides the most direct information on binding affinities. Experimental design was to immobilize to the CM5 chip, inject construct for test (at 10, 20 or 40 nM concentration), monitor loading, equilibration, and initial dissociation of construct, and then regenerate the chip with 10 mM HCl. After reequilibration with loading buffer, samples were retested. Data were evaluated using BIACORE software BIAevaluation VS 4.1. Data are presented in FIG. 5.

For the construct with a single maximum moiety the protocol worked well, data for four replicates were obtained and mean values with standard deviations for kon, koff and Kd were estimated. Values for all parameters for each value did not differ across concentration of construct tested, most probably due to the large variance (coefficient of variation 35-150%) in the individual measurements at the 10 and 20 nM concentrations. Data for the 40 nM data set were reproducible, and are summarized as mean+/−SD [N=4 replicates] for the single functional construct 1 as kon=16.5+/−0.6×10⁺⁴ M⁻¹ s⁻¹; koff=1.9=/−0.4×10⁻⁵ s¹; Kd=0.6+/−0.7 nM and for a native material lanalogous to that of Sjöbring U, Björck L, Kastern W, “Streptococcal protein G. Gene structure and protein binding properties,” J Biol Chem 266: 399-405 (1991) as kon=12.3+/−1.8×10⁺⁴M⁻¹ s⁻¹; koff=2.0=/−0.2×10⁺⁵ s⁻¹; Kd=0.7+/−0.7 nM. No statistical difference was noted in any of the parameters measured. Thus, the single maximum moiety version has a dissociation constant of ˜0.5 nM, and a half-life of 53,000 sec (1.5 hrs). Further, the tight binding with albumin can be disrupted by pH shift.

In contrast, the construct with a functional cassette (two maximum moieties) was very different in that binding was much tighter and binding could not be reversed with HCl treatment—and could be reversed with 6M guanidine HCl treatment but only with damage to the BIACORE chip and great technical difficulty in dealing with all the resultant salts. A limited number of replicates indicated binding features on the order of kon=3×10⁺⁵M⁻¹ s⁻¹; koff=2×10⁻⁹ s⁻¹; and Kd=5 pM. Thus, this molecule (with two maximum moieties) bound albumin much tighter than either of the other materials tested, with its half-life of 640,000,000 seconds' (which is to say, years') indicating irreversible binding of the type associated with the irreversible native avidin-biotin system.

In prior studies competition assays have been used to compare relative binding affinities. These assays have been shown to be highly dependent on assay conditions. There are two accepted approaches to extract data from such methods. Approach 1 seeks the answer to this question: What is the concentration of ligand needed to cause 50% inhibition of tracer to bound material? In this case, let the ratio of that binding feature be termed as R, where R=the ratio of (binding feature for a functional cassette with two maximum moieties/binding feature for a functional cassette with one maximum moieties). If no effect on binding, then R=1 and if increased binding is associated with multiple maximum moieties, then R<1. Approach 2 seeks the answer to another question: What is the rate of change (as concentration of ligand is varied) in binding of tracer to bound material around that 50% inhibition point? In this case, let the ratio of that binding feature be termed as S, where S=the ratio of (binding feature for a functional cassette with two maximum moieties/binding feature for a functional cassette with one maximum moieties). If no effect on binding, then S is=1 and if increased binding is associated with multiple domains, then S>1.

Conduct of such competition assays identify differences in relative affinities of each construct in a competitive ELISA patterned after that of Often RA and Boyle MDP, “Characterization of protein G expressed by human group C and G streptococci,” J. Microbiol. Methods 13:185-200 (1991). Triplicate wells were run for each treatment. After 60 minutes' incubation, the plates were washed and treated with streptavidin peroxidase and substrate to assess amount of biotinylated target in each well. After correction for blanks, data were normalized to absorbance at 450 nm for the zero binding protein treatment and expressed as “% inhibition” of that value.

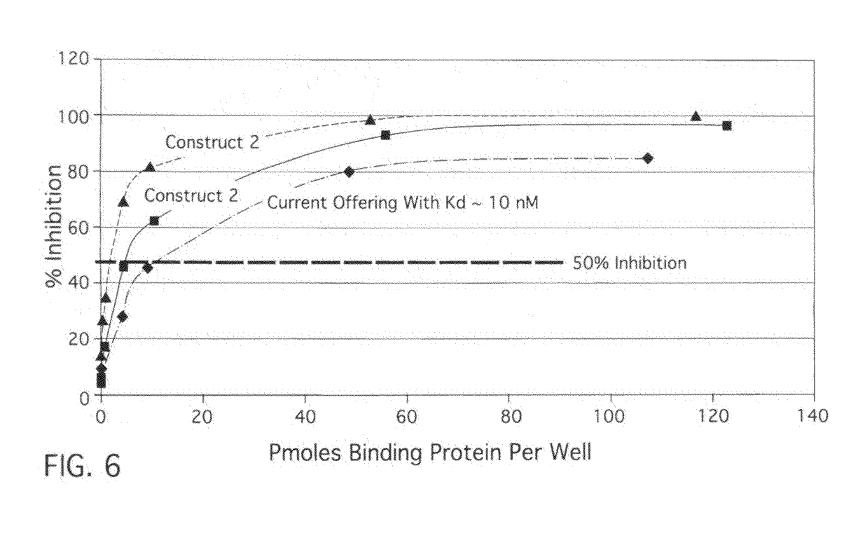

To provide a direct comparison to Illustration 1.1, the materials identical to that used in the Biacore assay (yielding the results of FIG. 5) were tested as Illustration 1.2 in a completion assay (FIG. 6). “Current offering” represents material analogous to that of Sjobring et al. (1991) with a reported Kd ˜1×10⁻⁹ M. Construct “2” is a functional cassette [with two maximum moieties] and represents the ultra-tight binding form. Concentrations [M] in the well can be estimated by pmoles/well divided by 2×E-4. In this assay apparent Kd (concentration needed for 50% inhibition) for one maximum moiety is 33 nM with rate of change of is 5 and respective values for a functional cassette with two maximum moieties 15 nM and 8.5. In this example, the value for R for the 50% inhibition point was 0.40 and for S the rate of change was 1.7. Thus, higher affinity was observed as measured with both parameters.

To illustrate the effect of changing the context (protein environment in which the maximum moiety and/or functional cassette is presented) an additional comparison was developed wherein the C-terminal segment was altered but all other aspects of the materials to be tested and the assay used remained as described above. For Illustration 1.3, apparent Kd (concentration needed for 50% inhibition) for one maximum moiety now was 25 nM with rate of change of is 3.5 and respective values for a functional cassette with two maximum moieties was 11 nM and 13. In this example, the value for R for the 50% inhibition point was 0.45 and for S the rate of change was 3.8. Thus, higher affinity was observed as measured with both parameters.

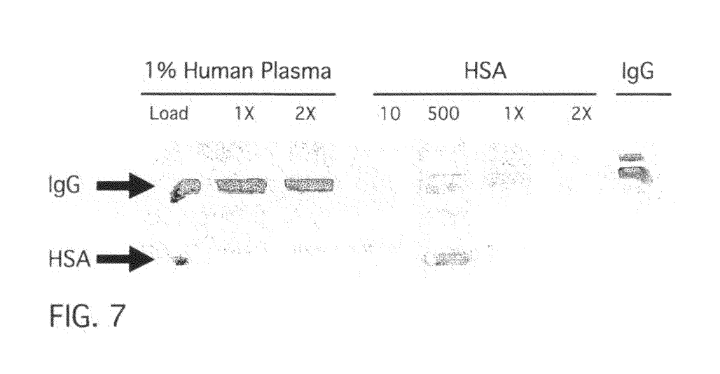

For Illustration 1.4, the materials used in illustration 1.3 were evaluated for ability to bind and remove albumin from complex samples. Human plasma (diluted to 1% v/v with PBS) and purified human serum albumin were treated with the functional cassettes attached to a silica surface, and then analyzed as follows. Separation was achieved under native (non-denaturing) conditions with SDS-PAGE using 12% Biorad Ready Gels and then transferred to nitrocellulose for ligand binding analysis. Native Protein G (bearing binding sites for both human serum albumin and IgG) was biotinylated for use as the probe, followed by immersing in streptavidin-HRP, and then peroxidase substrate. Blots were scanned and region of interest, without digital enhancement, in the region of ca 50-200 kD presented for review. The far left lane is human plasma diluted in saline to 1% (this should have human serum albumin at about 500 micrograms per mL) followed by an equal aliquot after either 1× or 2× treatment. The next four lanes represent purified human serum albumin presented at 10 micrograms/mL; 500 micrograms/mL (meant to approximate that in plasma sample analyzed), and then 500 micrograms/mL after either 1× or 2× treatment. Finally, position of an IgG [human immunoglobin G] standard is shown. Samples of 1% human blood plasma, 500 and 10 μgm/mL human serum albumin were prepared. The construct with two maximum moieties was attached to a silica surface. Duplicate samples were processed (termed 1×) and pooled. Then, an aliquot was processed a second time (termed 2×). Equal aliquots of all samples were separated by SDS-PAGE and ligand blot assay to provide a semi-quantitative estimate of both human serum albumin and IgG on the blot (FIG. 7). Virtually all of the material migrating in the region of human serum albumin was removed in the 1× treatment, with little-to-no additional effect with the second treatment. For purified human serum albumin, a small amount of cross reading material (equivalent to the 10 μgm/mL sample) was noted that was removed with the 2× treatment. These representative data suggest that about 98% of the human serum albumin (presented either in diluted plasma or purified human serum albumin) can be removed in a few minutes with a single treatment.

In Illustration 1.5, a direct analysis using the same immobilized proteins used for Illustration 1.1 but with a totally different assay protocol was employed: flow cytometry. Immobilized binding proteins with either a functional cassette with two maximum moieties or one maximum moieties were place on separate beads and their binding studied with fluorescein labeled albumin. The apparent Kd for the functional cassette with a single maximum moiety was 240 nM while that for the functional cassette with two maximum moieties was 160 nM. Let the ratio of that binding feature be termed as F, where F=the ratio of (functional cassette with two maximum moieties/functional cassette with one maximum moiety). If no effect on binding, then F=1 and if increased binding is associated with multiple domains, then F<1. The F value was 0.67. Thus, higher affinity was observed.

To summarize, the binding of albumin was evaluated as a single maximum moiety or in a functional cassette with two maximum moieties, and evaluated in several protein contexts and three different types of assays. While the resultant numbers used to approximate Kd differ, the binding affinity for a functional cassette was always stronger than for the single maximum moiety and the material has excellent properties for extraction of albumin from complex mixtures.

In example 2, using the overview outlined in FIGS. 2-4, a different functional cassette was generated to extract IgG from complex mixtures. For this case the maximum moiety has three minimum binding domains leading to functional cassettes with two maximum (six minimum moieties) moieties.

The DNA and protein structures of the maximum moiety for IgG binding are provided as follows (SEQ ID NO. 3).

LGIDPFTALPKTD

[symbol]

[symbol]

[symbol]

[symbol]

KPEVIDASELTPAVT

[symbol]

[symbol]

[symbol]

[symbol]

KPEVIDASELTPAVT

[symbol]

[symbol]

[symbol]

[symbol]

[symbol]

MVTESRALPKTD

[symbol]

[symbol]

[symbol]

[symbol]

[symbol]

KPEVIDASELTPAVT

[symbol]

[symbol]

[symbol]

[symbol]

[symbol]

[symbol]

KPEVIDASELTP
AVT

[symbol]

[symbol]

[symbol]

[symbol]

[symbol]

[symbol]

K
GELKLCITGDALVALPEGES

The amino acids presented in bold print are sequences binding IgG (note different amino acid sequences), while those in regular case capitals are spacer regions. The DNA sequence covering the same regions is ctg gga att gat ccc ttc acc gca tta cct aag act gac act tac aaa tta atc ctt aat ggt aaa aca ttg aaa ggc gaa aca act act gaa get gtt gat get get act gca gaa aaa gtc ttc aaa caa tac get aac gac aac ggt gtt gac ggt gaa tgg act tac gac gat gcg act aag acc ttt aca gtt act gaa aaa cca gaa gtg atc gat gcg tct gaa tta aca cca gcc gtg aca act tac aaa ctt gtt att aat ggt aaa aca ttg aaa ggc gaa aca act act gaa get gtt gat get get act gca gaa aaa gtc ttc aaa caa tac get aac gac aac ggt gtt gac ggt gaa tgg act tac gac gat gcg act aag acc ttt aca gtt act gaa aaa cca gaa gtg atc gat gcg tct gaa tta aca cca gcc gtg aca act tac aaa ctt gtt att aat ggt aaa aca ttg aaa ggc gaa aca act act aaa gca gta gac gca gaa act gca gaa aaa gcc ttc aaa caa tac get aac gac aac ggt gtt gat ggt gtt tgg act tat at gat gat gcg act aag acc tit acg gta act gaa atg gtt aca gag tct aga gca tta cct aag act gac act tac aaa tta atc ctt aat ggt aaa aca ttg aaa ggc gaa aca act act gaa get gtt gat get get act gca gaa aaa gtc ttc aaa caa tac get aac gac aac ggt gtt gac ggt gaa tgg act tac gac gat gcg act aag acc ttt aca gtt act gaa aaa cca gaa gtg atc gat gcg tct gaa tta aca cca gcc gtg aca act tac aaa ctt gtt att aat ggt aaa aca ttg aaa ggc gaa aca act act gaa get gtt gat get get act gca gaa aaa gtc ttc aaa caa tac get aac gac aac ggt gtt gac ggt gaa tgg act tac gac gat gcg act aag acc ttt aca gtt act gaa aaa cca gaa gtg atc gat gcg tct gaa tta aca cca gcc gtg aca act tac aaa ctt gtt att aat ggt aaa aca ttg aaa ggc gaa aca act act aaa gca gta gac gca gaa act gca gaa aaa gcc ttc aaa caa tac get aac gac aac ggt gtt gat ggt gtt tgg act tat at gat gat gcg act aag acc ttt acg gta act gaa atg gtt aca gag aag ggc gag ctc aag ctt tgc atc acg gga gat gca cta gtt gcc cta ccc gag ggc gag tcg (SEQ ID NO. 4).

In Illustration 2.1 the maximum moiety was compared with the functional cassette. In the assay, the immobilized binding protein on the plate was a functional cassette with two maximum domains and biotinylated IgG. Apparent Kd (concentration needed for 50% inhibition) for one maximum domain was 1.7 nM with a rate of change of 31 and the respective values for a functional cassette with two maximum moieties was 1 nM and 50. In this example, the value for R for the 50% inhibition point was 0.60 and for S the rate of change was 1.6. Thus, higher affinity was observed as measured with both parameters.

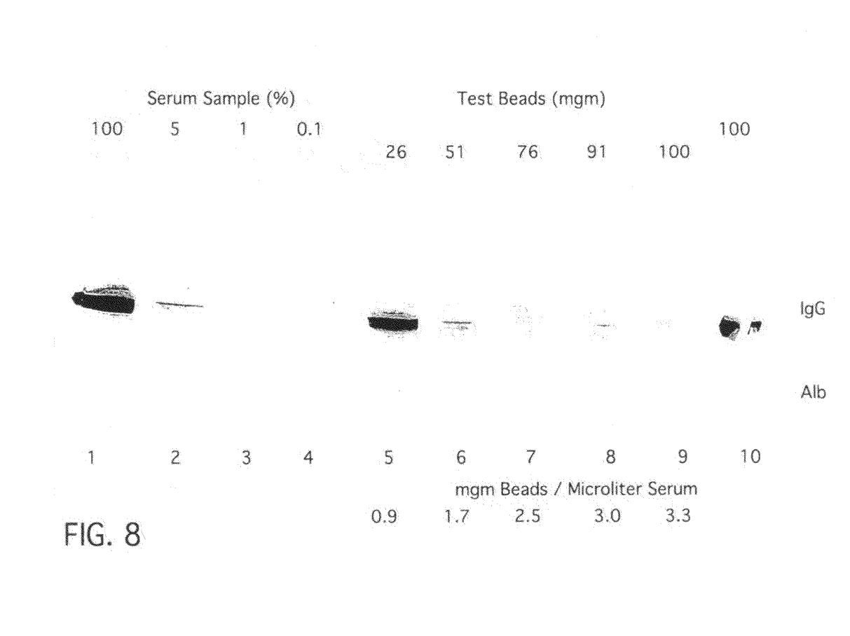

In Illustration 2.2, utility of the material according to the invention was demonstrated as FIG. 8. Human serum (30 microliters) was diluted to 500 microliters, and then treated with indicated amount of silica beads coated with the functional cassette containing two maximum domains specific for IgG. Equivalent aliquots of stock sample (100, 5, 1 and 0.1%) and treated samples were processed for comparison. Standards of albumin and IgG are indicated. In this example, 1.7 mgs of coated beads were required to extract >99% of the IgG from the sample. The functional cassette with two maximum domains was both effective and specific in removing IgG from the human serum sample.

In Illustration/example 3, a heterogeneous system was assembled where the functional cassette was a combination of functional cassettes for both albumin and IgG.

In Illustration 3.1 the same material as Illustration/example 3 was immobilized on plate, and tested with individually with both albumin and IgG. The 50% inhibition point for albumin was 6 nM and slope was 8.8; for IgG it was 1 nM with a slope of 44. This establishes that complex functional cassettes can be assembled with maximum moieties for different targets and their properties as predicted from studies of individual functional cassettes.

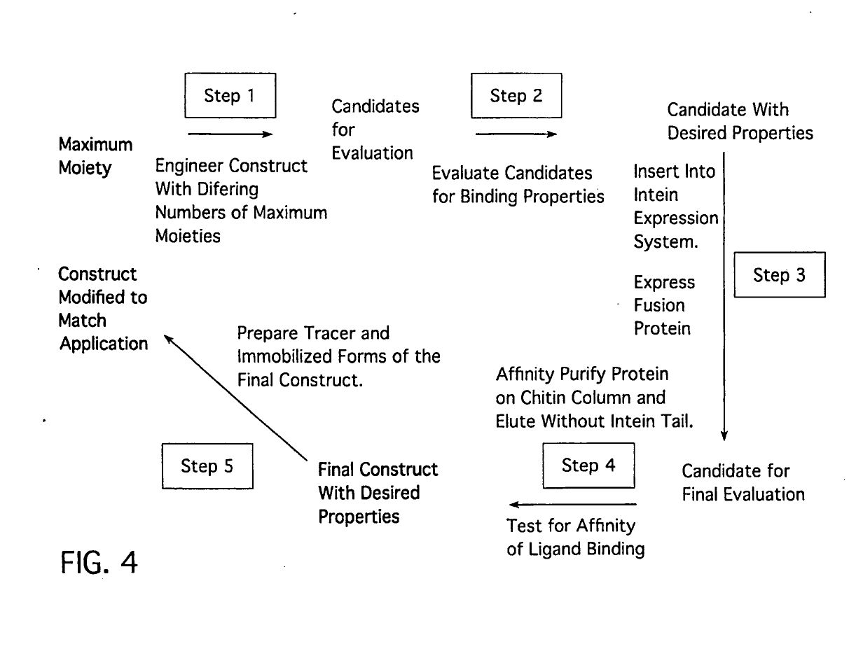

In Illustration 4, it is apparent that the approach of FIGS. 2-4 can be generalized to create a wide variety of functional cassettes to generate a useable material for extracting a wide variety of materials. A few of many examples are provided to introduce maximum moieties with selectivity for human IgG3 (Podblieiski A, Hawlitzky J, Pack T D, Flosdorff A and Boyle MDP, “A group A streptococcal Enn protein potentially resulting from intergenomic recombination exhibits atypical immunoglobulin-binding characteristics,” Molecular Microbiology, 12: 725-736 (1994)) or all four human IgG subclasses (Sjöbring et al. (1991)), or for human IgA (Bessen D E, “Localization of immunoglobulin A-binding sites within M or M-like proteins of group A streptococci,” Infect Immun. 62: 1968-1974 (1994)). The process presented in FIG. 4 teaches how to take these maximum moieties and move them to ultra-high affinity functional cassettes.

It is well known that functional cassettes of the sort described herein can be combined, with or without chemical activation and contained in a variety of contexts, including but not limited to detectable elements and solid surfaces, for use as an affinity reagent for the analysis of a variety of biological media or samples. Such uses can be conducted with robotic or manual methods. In addition, functional cassettes with or without their cognate target molecule, can be used as a transforming agent for a variety of purposes. Further, functional cassettes can be used alone or in context with other materials for therapeutic purposes in a variety of in vivo and in vitro situations.

It is noteworthy that nature teaches the ability to modify affinities of receptor molecules by covalent or non-covalent association of similar or dissimilar polypeptide e.g. association of immunoglobulin heavy and light chains, association of zeta chains to cytokine receptors or the non covalent association of identical subunits, e.g. avidin. In the method taught herein the ultra-high affinity binding functional cassette is achieved as a single contiguous primary amino acid sequence within a single polypeptide chain.

The following definitions are helpful in understanding the present invention.

As used herein, the term “bacteria” is defined as a unicellular micro-organism.

As used herein, the term “virus” is defined as a microscopic infectious agent that can reproduce only inside a host cell.

As used herein, the term “fungal” is defined as any member of a large group of eukaryotic organisms that includes organisms such as yeasts and molds.

As used herein, the term “archaea” is defined as single-celled organisms without nuclei and with membranes different from all other organisms.

As used herein, the term “source organism” is a defined microbes, viruses, fungi or archaea.

As used herein, the term “spacer” is a sequence of five to forty amino acids, not constituting a functional cassette, that may be incorporated between or at the termini of a sequence of amino acids of interest.

As used herein, the term “functional cassette” is defined as a plurality of maximum moieties, which may be separated by spacers, and may have termini that provide additional valuable features, but not limited to, aspects such as optimization of manufacture by recombinant technologies, increased solubility of final protein, enhanced and oriented binding to solid surfaces, or simplification of the processes needed in the formation of detectable labeled materials.

As used herein, the term “maximum moiety” is defined as a description of biological activity related to the minimal sequence that accounts for all the binding activity of the intact protein for the target ligand as isolated from the source organism. The maximum moiety may contain individual protein domains that differ in binding activity and are separated by spacers. The maximum moiety will contain between 50 and 500 amino acids, preferably between 50 and 250 amino acids.

As used herein, the term “minimum moiety” is defined as a description of biological activity related to the minimal contiguous sequence that bears binding activity of the intact protein for the target ligand and is used for subsequent in vitro manipulations to alter binding affinity. The minimum moiety will contain between 10 and 24 amino acids.

As used herein, the term “duplicate” is defined as making copies of a functional cassette, such copies may or may not include identical or non-identical spacers.

As used herein, “context” is defined as the amino acid sequences that precede or follow the binding domain and which may influence its properties.

As used herein, the term “receptor” generally refers to one member of a pair of compounds that specifically recognize and bind to each other. The other member of the pair is termed a “ligand” and includes such things as complexes, protein-protein interactions, multianalyte analyses, and the like. Receptor/ligand pairing may include protein receptor (membranous), and its natural ligand (associated, or other proteins or small molecules or sugars). Receptor/ligand pairs may also include antibody/antigen binding pairs, lectin sugar binding pairs, cellular surface protein/soluble molecule binding pairs, complementary nucleic acid, nucleic acid associating proteins and their nucleic acid ligands such as aptamers and their proteins, metal chelators and metal binding protein ligands, mimic dyes and their protein ligands, organic molecules and their interaction, such as hydrophobic patches, on or with biomolecules, ion exchangers and their electrostatic interaction on or with biomolecules, and the like.

As used herein, the term “recombinant” is used to describe non-naturally altered or manipulated nucleic acids, host cells transfected with exogenous nucleic acids, or polypeptides expressed non-naturally, through manipulation of isolated nucleic acid, especially DNA and transformation of host cells. Recombinant is a term that specifically encompasses nucleic acid molecules that have been constructed in vitro using genetic engineering techniques, and use of the term “recombinant” as an adjective to describe a molecule, construct, vector, cell, peptide, or polynucleotide specifically excludes naturally occurring molecules, constructs, vectors, cells, polypeptides or polynucleotides.

As used herein, the term “binding” refers to the determination by standard techniques that a binding moiety or functional cassette recognizes and binds reversibly to a given target. Such standard techniques to detect or measure serum albumin binding include ELISA, equilibrium dialysis, gel filtration, and the monitoring of spectroscopic changes that result from binding, e.g., using fluorescence anisotropy, either by direct binding measurements or competition assays with another binder.

As used herein, the term “binding constant” is defined as an equilibrium constant and relates to the relationship [A+B→AB] where A and B are two species that can interact in a non-covalent manner to create a complex and the dissociation constant Kd=[A] [B]/[AB] has the units of M. Either A or B can be more complex than a single molecule.

As used herein, the term “off-rate” is defined as the first order reaction related to the breakup of the AB complex as defined for the binding constant. The velocity of the reverse reaction, in which the AB complex dissociates, is the product of [AB] and koff, which has units of (sec)-1.

As used herein, the term “ultra-high binding” affinity refers to a binding affinity of the order approaching that for strepatavidin and biotin or avidin and biotin with a Kd below 1E-11M. As used herein, the term “high binding” affinity refers to a binding affinity on the order seen for a polyclonal antibody and its cognate antigen.

As used herein, the term “specificity” refers to a binding moiety or functional cassette having a higher binding affinity for one target over another. As one example of many, the term “human serum albumin specificity” refers to a binding moiety having a higher affinity for human serum albumin as compared with other proteins [i.e. other serum proteins such as fibrinogen] or ovalbumin. Preferred binding moieties described herein will have at least a 100-fold greater affinity for their cognate ligand than other serum proteins.

As used herein, the term “polypeptide” refers to a polymer comprising two or more amino acid residues linked with amide bonds, and the term “peptide” is used herein to refer to relatively short polypeptides, e.g., having fewer than about 30 amino acids. The term “polypeptide” also encompasses the term “protein”.

As used herein, the term “detectably labeled” is to be understood as describing linking the binding moiety to a compound, or “label”, such as a dye [such as fluorescein]; a radionuclide, such as iodine 131-I or a technetium [99-Tc]-containing compound; an enzyme [such as horseradish peroxidase]; or a detectable metal [such as a paramagnetic ion], wherein the label thereafter provides a signal that can be detected by some appropriate means. The term “detectably labeled” also includes incorporating into a molecule detectable radioactive atoms [such as 32-P, 35-S, or 14-C] in place of a non-radioactive isotope of the same element. “Detectably labeled” also refers to any molecule that is linked or bound to one of a pair of binding partners, whereby detection of the linked [i.e., labeled] molecule is made when the binding partners form a complex. Many such pairs of binding partners are used in standard detection systems known in the art, such binding partners include, without limitation, biotin and streptavidin [either of which may also be conjugated to an enzyme, such as HRP or beta-galactosidase, which in turn can be used in a reaction to generate a detectable signal], antibody and epitope binding partners [including epitopes present on the molecule to be detected], and enzyme and substrate binding partners.

As used herein, “solid surfaces” may be fabricated from, but not limited to, metals, crystals, glasses, plastics, polymers, composites, other useful materials or combinations thereof. For example, these glasses may be silica glasses, borosilicates, sodium borosilicates, and other useful materials.

As used herein, an “affinity reagent” refers to a chemical or biological species that can be bound to the solid surface and which has an affinity for an analyte or another affinity receptor.

As used herein, an “analyte” refers to molecule(s) of interest present in a biological sample. Analytes may be, but are not limited to, nucleic acids [DNA, RNA], peptides, hormonal peptides, polypeptides, proteins, lipoproteins, glycoproteins, protein complexes, carbohydrates or small inorganic or organic molecules having biological function. Analytes also includes cells and cellular organelles. Analytes may naturally contain sequences, motifs or groups recognized by the affinity reagents or affinity receptors or may have these recognition moieties introduced to them via processing such as cellular, extracellular, enzymatic, chemical, and the like.

As used herein, an “affinity receptor” refers to atomic or molecular species having an affinity towards molecules of biological function. Affinity receptors may be organic, inorganic or biological by nature and can exhibit broad [targeting numerous analytes] or narrow [target a single analyte] specificity. Examples of affinity receptors include, but are not limited to, antibodies, antibody fragments, synthetic paratopes, peptides, polypeptides, enzymes, lectins, proteins, multi-subunit protein receptors, mimics, organic molecules, polymers, inorganic molecules, chelators, nucleic acids, aptamers, either as purified or semi-purified entities or associated with cells or organelles.

As used herein, “biological media” or “biological sample” refers to a fluid or extract having a biological origin. Biological media may be, but are not limited to, cell extracts, nuclear extracts, cell lysates and excretions, blood, sera, plasma, urine, sputum, sinovial fluid, cerebral-spinal fluid, tears, feces, saliva, membrane extracts and the like.

As used herein, “chemically activate” refers to the process of exposing the affinity reagent to chemicals [or light] in order to subsequently attach [or photoactivate] tethering linkers and affinity receptors. Compounds able to activate affinity reagents may be, but are not limited to organic, inorganic or biological reagents. Often, it is advantageous to activate the affinity reagent using multiple steps including the use of a tethering linker. As used herein, “tethering linker” refers to compounds intermediate to the affinity reagent and the affinity receptor that exhibit the desirable characteristics of being able to be derivatized with high densities of affinity receptor and showing low binding of non-specified compounds. The tethering linker may be intrinsically active or require activation for attachment. Suitable tethering compounds include but are not limited to homo/hetero functional organics, natural and synthetic polymers and biopolymers.

As used herein, “analysis” refers to the ascertainment of the kind or amount of one or more of the constituents of materials, whether obtained in separate form or not. The method of ascertainment can be chemical, physical, or biological.

As used herein, “robotic” refers to devices and procedures capable of the unattended processing of samples. Preferably, the robotic operates on numerous samples in parallel to maximize the number of samples processed and analyzed in a given amount of time.

As used herein, “transform” refers to alteration mediated by the selected input material.

As used herein, “transforming agent” refers to a chemical, physical or biological material(s) that is responsible for the transformation.

As used herein, “therapeutic” refers to having or exhibiting healing powers or relating to medical treatment of a disease or condition.

The following provides some additional commentary concerning the present invention.

In the context of the present invention, the binding moieties disclosed herein may be advantageously linked to other compounds, such as diagnostic reagents, therapeutic polypeptides or other drugs, for example to give such compounds improved affinity for serum albumin or IgG. In this context, the term “linked” is a broad term encompassing any suitable means of attaching or conjugating the compound of interest to a binding moiety of this invention. Many suitable linking means are known in the art and include but are not limited to covalent conjugation, chemical cross-linking via heterobifunctional or homobifunctional cross-linking agents, designing of fusion proteins by linking encoding polynucleotides for the fusion partners [i.e., the binding moiety and a polypeptide of interest] together in-frame for expression of the fused polypeptide, affinity linking such as biotinylation [i.e., for linking to a streptavidin-bearing substrate], ionic links, or any other means by which two or more separate entities may be bound or aggregated to form a single entity or complex.

The preferred embodiments of the invention are described above. While these descriptions directly describe the above embodiments, it is understood that those skilled in the art may conceive modifications and/or variations to the specific embodiments shown and described herein. Any such modifications or variations that fall within the purview of this description and the accompanying claims are intended to be embraced by this patent specification. Unless specifically noted, it is the intention of the inventors that the words and phrases in the specification and claims be given the ordinary and accustomed meanings to those of ordinary skill in the applicable art(s). The foregoing description of preferred embodiments and the best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiments was chosen and described in order to best explain the principles of the invention and its practical application and to enable others skilled in the art to best utilize the invention in various embodiments and various modifications as are suited to the particular use contemplated.

1. A method of making functional cassettes, comprising: (a) identifying a maximum moiety of interest having between 50 and 500 amino acids therein; (b) replicating a peptide or protein encoding said maximum binding moiety at least two times, to create at least two binding sequences; and (c) coupling said binding sequences into the same polypeptide wherein each of said binding sequences is separated by between five and forty amino acids; wherein said polypeptide has a geometrically increased binding affinity compared to the arithmetically summed binding affinities of said individual maximum moieties and further wherein said geometrically increased binding affinity yields a binding constant [Kd] of less than 1E-11 M and with a half life of at least 36,000 seconds when the binding constant is determined by surface plasmon resonance assessment in real time.

2. A purified molecule containing two maximum moieties having between 50 and 500 amino acids embodied in two respective binding sequences, with the binding sequences' being coupled into the same polypeptide and separated by between five and forty amino acids, such that the molecule has a binding constant [Kd] of less than 1E-11M with a half life of at least 36,000 seconds when the binding constant is determined by surface plasmon resonance assessment in real time.

3. The method according to claim 1 wherein steb b), replicating a peptide or protein encoding said maximum binding moiety at least two times, to create at least two binding sequences, is repeated so as to replicate a peptide or protein encoding a maximum moeity at least three times, to create at least three binding sequences.

4. The method according to claim 1 wherein step b), replicating a peptide or protein encoding said maximum binding moiety at least two times, to create at least two binding sequences, is repeated so as to replicate a peptide or protein encoding a maximum moeity at least four times, to create at least four binding sequences.

5. The purified molecule according to claim 2, wherein said molecule contains 3 maximum moieties.

6. The purified molecule according to claim 2, wherein said molecule contains at least 4 maximum moieties.
